(19) United States
(12) Patent Application Publication
(10) Pub. No.: US 2022/0226006 A1
(43) Pub. Date: Jul. 21, 2022

(54) Surgical Tool and Method

(71) Applicant: Daniel J. Olson, Erie, PA (US)

(72) Inventor: Daniel J. Olson, Erie, PA (US)

(21) Appl. No.: 17/607,894

(22) PCT Filed: May 5, 2020
(86) PCT No.: PCT/US20/31427

§ 371 Date: Oct. 31, 2021

Related U.S. Application Data
(60) Provisional application No. 62/843,453, filed on May 5, 2019.
(60) Provisional application No. 62/908,562, filed on Sep. 30, 2019.

(51) Int. Cl. A61B 17/17; A61B 17/88

(52) U.S. Cl. CPC A61B 17/1775; A61B 2017/565; A61B 17/8866

(57) ABSTRACT

A surgical tool and method for engaging and manipulating a bone includes a tool body having a main body axis, a threaded end, and a handling end, the threaded end having threads and a tool stop. The handling end is positioned to allow a user to grasp, manipulate, and rotate the tool body around the main body axis. The threads are positioned to allow the surgical tool to bore into and engage the bone when the tool body is rotated in a boring direction around the main body axis, the threads allowing for tightening engagement with the bone as the tool body is rotated in the boring direction around the main body axis. The tool stop is positioned to prevent the tool from being further rotated around the main body axis to prevent further boring into the bone when the tool stop contacts the bone.

### BACKGROUND

As modern surgical techniques become increasingly less invasive, surgeons must often contend with tight spaces created by minimal surgical incisions and surrounding anatomical structures. In surgeries involving the manipulation of bone, anatomical structures such as tendons, muscle, skin, and adjacent bone are often tightly packed in and around the bone, requiring a surgeon to exert significant linear or rotational forces against the bone to move, adjust, or rotate the bone during the procedure.

For example, in the case of podiatric surgeries, surgeons must often move, adjust, or rotate bones of the foot along one or more defined planes. As shown in the context of a left human foot 2 in FIG. 1, these include the saggital plane 4, transverse plane 6, and frontal plane 8. Comparing FIG. 1 with FIG. 2, which depicts a normal skeletal arrangement of a left foot 10, each metatarsal bone 12, proximal bone 14, and distal bone 16 lies within the saggital, transverse, and frontal planes 4, 6, and 8.

The need for linear and rotational bone movement across the perpendicular planes is apparent in the specific case of bunion correction surgery. FIG. 3 depicts the left foot 10 of FIG. 2 after the foot 10 has developed a bunion condition. As best understood by comparing the normal skeletal arrangement in FIG. 2 with the skeletal arrangement after development of the bunion condition in FIG. 3, an increased spacing 18 develops between the first metatarsal bone 20 and second metatarsal bone 22. This is partially due to counterclockwise rotation of the first metatarsal bone 20 and its associated pair of sesamoids 24.

An objective in performing corrective surgery for the bunion condition depicted in FIG. 3 is best understood by comparing FIGS. 1-3 with the desired corrections best understood with reference to FIGS. 4A-C. A front cross sectional view of the normal skeletal arrangement 10 of the left foot of FIG. 2 taken along an approximately frontal plane at the first metatarsal head 26 is depicted in FIG. 4A. In this view, FIG. 4A depicts the first metatarsal bone 20 and its first metatarsal head 26 correctly positioned with the associated sesamoids 24 also correctly positioned just beneath the first metatarsal head 26.

Referring now to FIG. 4B, a similar front cross sectional view of the skeletal arrangement 10 is depicted after development of the bunion condition of FIG. 3. In this view, FIG. 4B depicts the first metatarsal bone 20 and its first metatarsal head 26 rotated in a counterclockwise direction, along with the sesamoids 24 which have rotated along with the first metatarsal head 26 toward the increased spacing 18 that has developed between the first and second metatarsal bones 20 and 22.

Now referring to FIG. 4C, which is a further front cross sectional view of the skeletal arrangement 10 of FIGS. 2 and 3, a desired objective of corrective bunion surgery is depicted with clockwise swivel or rotation 28 of the first metatarsal head 26 and sesamoids 24 from the bunion condition positions depicted in FIG. 4B back to positions similar to the normal positions depicted in FIG. 4A.

Although bunion correction surgery is used herein as an example, surgeons must also contend with similar surgical constraints when operating on bone in other parts of both human and animal anatomy, requiring analogous bone rotations, adjustments, and manipulations within and across defined anatomical planes in other anatomical regions

### SUMMARY OF THE INVENTION

A surgical tool and method for engaging and manipulating a bone includes a tool body having a main body axis, a threaded end, and a handling end, the threaded end having threads and a tool stop. The handling end is positioned to allow a user to grasp, manipulate, and rotate the tool body around the main body axis. The threads are positioned to allow the surgical tool to bore into and engage the bone when the tool body is rotated in a boring direction around the main body axis, the threads allowing for tightening engagement with the bone as the tool body is rotated in the boring direction around the main body axis. The tool stop is positioned to prevent the tool from being further rotated around the main body axis to prevent further boring into the bone when the tool stop contacts the bone. The surgical tool thereby allows for manipulation, movement, and rotation of the bone with the surgical tool when the tool is in tightened engagement with the bone.

In some embodiments, a guide hole extends through the tool body substantially along the main body axis. The guide hole allows a positioning guide to extend at least partially through the tool body and into a bone at a bone boring position to align and guide the threads of the surgical tool to the bone boring position.

### BRIEF DESCRIPTION OF THE DRAWINGS

For a more complete understanding and appreciation of this invention, and its many advantages, reference will be made to the following Detailed Description taken in conjunction with the accompanying drawings.

FIG. 1 depicts defined cardinal planes of a left human foot;

FIG. 2 depicts a front perspective view of a normal skeletal arrangement of a left human foot:

FIG. 3 depicts a front perspective view of the skeletal arrangement of the left human foot of FIG. 2 after development of a bunion condition;

FIG. 4A front depicts a cross sectional view of the normal skeletal arrangement of FIG. 2 taken along an approximately frontal plane at the first metatarsal head;

FIG. 4B depicts a cross sectional view of the skeletal arrangement of FIG. 2 after development of, a bunion condition;

FIG. 4C depicts a cross sectional view of the skeletal arrangement of FIG. 2 during correction of a bunion condition;

FIG. 5A depicts a front perspective view of a surgical tool according to one embodiment of the invention;

FIG. 5B depicts a front cross sectional view of the surgical tool of FIG. 5A taken along line 5B-5B of FIG. 5A;

FIG. 6A depicts a magnified front view of the threaded end of the surgical tool of FIG. 5A.

FIG. 6B depicts a magnified front view of the threaded end of a surgical tool according to one embodiment of the invention;

FIG. 7 depicts a front perspective view and magnification of the skeletal arrangement of FIG. 3 after the insertion of a guide wire in the first metatarsal head;

FIG. 8 depicts a perspective view of the skeletal arrangement of FIG. 4 with the addition of a surgical tool of the invention to the guide wire;

FIG. 9 depicts a perspective view and magnification of the skeletal arrangement of FIG. 8 with the surgical tool positioned at a boring position;

FIG. 10 depicts a perspective view and inset view of the skeletal arrangement of FIG. 10 with the tool bored into and in threaded engagement with the first metatarsal head;

FIG. 11A depicts a front perspective view of a surgical tool according to one embodiment of the invention;

FIG. 11B depicts a front cross sectional view of the surgical tool of FIG. 11A taken along line 11B-11B of FIG. 11A;

FIG. 12A depicts a front view of a surgical tool according to one embodiment of the invention;

FIG. 12B depicts a front perspective view of the surgical tool of FIG. 12A;

FIG. 13A depicts a front view of a surgical tool according to one embodiment of the invention;

FIG. 13B depicts a front perspective view of the surgical tool of FIG. 13A;

FIG. 14 depicts a front perspective view of a surgical tool according to one embodiment of the invention;

FIG. 15 depicts a front perspective view of a surgical tool according to one embodiment of the invention;

FIG. 16 depicts a front perspective view of a surgical tool according to one embodiment of the invention;

FIG. 17 depicts a front perspective view of a surgical tool according to one embodiment of the invention;

FIG. 18 depicts a front perspective view of a surgical tool according to one embodiment of the invention;

FIG. 19 depicts a front perspective view of a surgical tool according to one embodiment of the invention;

FIG. 20 depicts a front perspective view of a surgical tool according to one embodiment of the invention;

FIG. 21 depicts a front perspective view of a surgical tool according to one embodiment of the invention;

FIG. 22 depicts a front perspective view of a surgical tool according to one embodiment of the invention;

FIG. 23A depicts a front view of a surgical tool according to one embodiment of the invention;

FIG. 23B depicts a front cross sectional exploded view of a surgical tool according to one embodiment of the invention;

FIG. 24A depicts a front perspective view of a surgical tool according to one embodiment of the invention;

FIG. 24B depicts a front view of a surgical tool according to one embodiment of the invention;

FIG. 25A depicts a perspective view of a wire angulation tool according to one embodiment of the invention; and

FIG. 25B depicts a side view of the wire angulation tool of FIG. 25A positioned on a left foot to align a guide wire into a first metatarsal head according to one embodiment of the invention.

### DETAILED DESCRIPTION OF THE INVENTION

Referring to the drawings, similar reference numerals are used to designate the same or corresponding parts throughout the several embodiments and figures. In some drawings, some specific embodiment variations in corresponding parts are denoted with the addition of lower case letters to reference numerals.

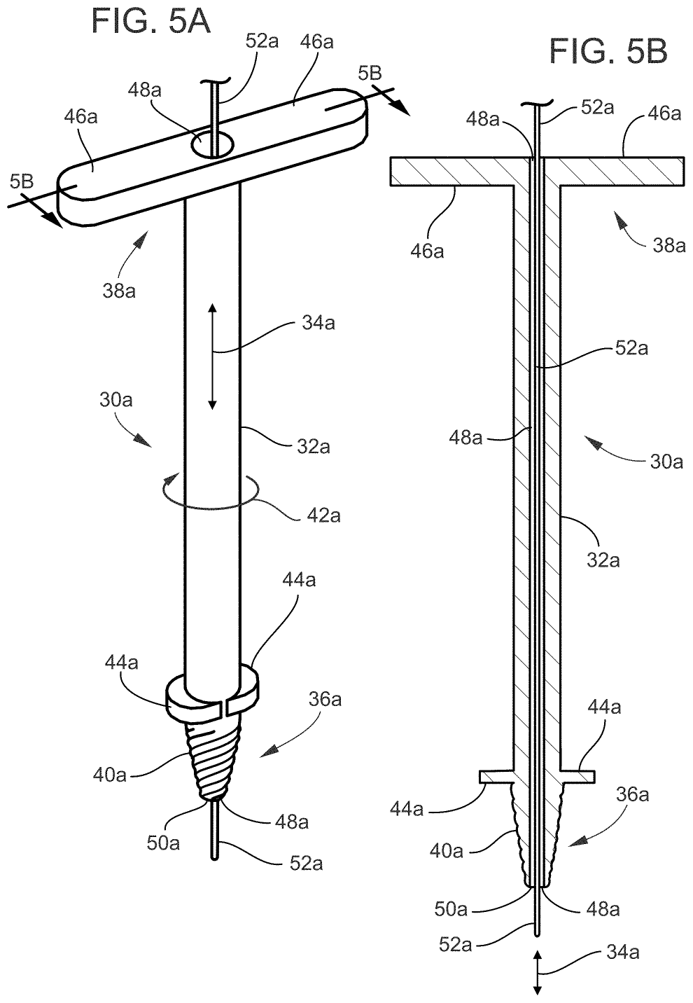

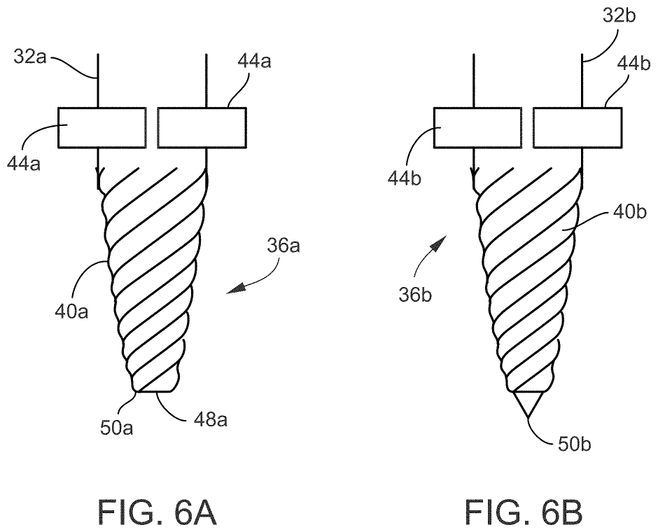

FIG. 5A depicts a perspective view of a surgical tool 30a of the invention comprising a tool body 32a having a main body axis 34a, a threaded end 36a, and a handling end 38a. Conically shaped, clockwise boring threads 40a are positioned at the threaded end 36a and are oriented to drill or bore into bone material when the surgical tool 30a is rotated around the main body axis 34a in a right and clockwise boring direction 42a. The threaded end 36a also includes a tool stop 44a comprising two small tabular legs extending outwardly from the tool body 32a and just slightly above the threads 40a. At the handling end 38a, two handles 46a also extend outwardly from the tool body 32a. The handles 46a allow the user to grasp, manipulate, and rotate the tool body 32a around the main body axis 34a. The two handles 46a, the tool body 32a, and the outward extending dimensions of both legs of the tool stop 44a all lie substantially within a plane shared with the main body axis 34a wherein the surgical tool 30a remains substantially flat when placed on a flat surface (not shown). Although it is contemplated the surgical tool 30a would typically be 4 to 5 inches in length, it is further contemplated this would vary greatly and ultimately depend on the specific application or surgical circumstances.

A front cross sectional view of the surgical tool 30a taken along line 5B-5B of FIG. 5A is depicted in FIG. 5B. As best understood by comparing FIGS. 5A and 5B, the surgical tool 30a is cannulated with the inclusion of a guide hole 48a extending substantially along the main body axis 34a, the guide hole 48a opening at the threaded end 36a at the tip 50a of the threads 40a and extending to the handling, end 38a, opening at a position between the handles 46a. As best understood with brief reference to the magnified view of the threaded end 36a of the surgical tool 30a in FIG. 6A, the positioning of the guide hole 48a at the tip 50a of the threads 48a causes the tip 50a to be slightly blunted as shown.

Although the invention has been shown, and described as including a blunt thread tip, it will be appreciated that other tip configurations are also possible within the contemplated scope of the invention. For example, FIG. 6B depicts a magnified view of a threaded end 36b of a contemplated surgical tool having a tool body 32b, tool stop 44b and clockwise boring threads 40b similar to the surgical tool of 30a of FIG. 6A. However, being non-cannulated, the surgical tool of FIG. 6B lacks a guide hole and therefore includes a pointed tip 50b at bottom of the threads 40b, possibly enhancing the bone boring capability of threads 40b in some applications.

Referring again to the embodiment surgical tool 30a of FIGS. 5A-6A, the guide hole 48a allows for the accommodation of a positioning guide that is a guide wire 52a. Since the guide hole 48a is open at both the tip 50a of the threads 48a and also between the two handles 46a, the surgical tool 30a allows the guide wire 52a to extend completely though the surgical tool 30a along the complete length of the main body axis 34a as shown in FIGS. 5A and B.

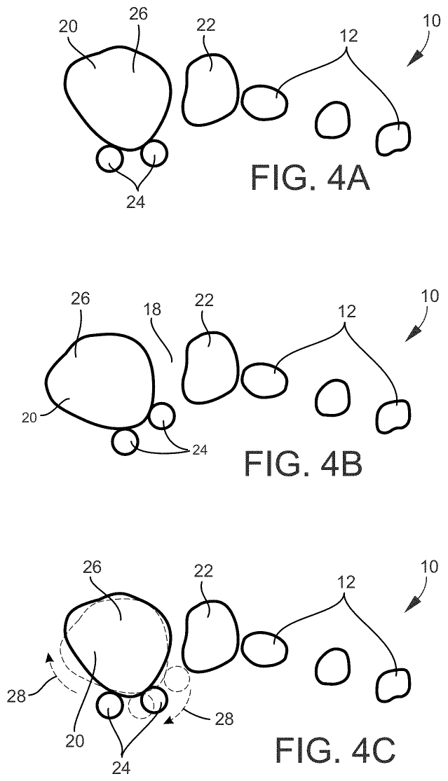

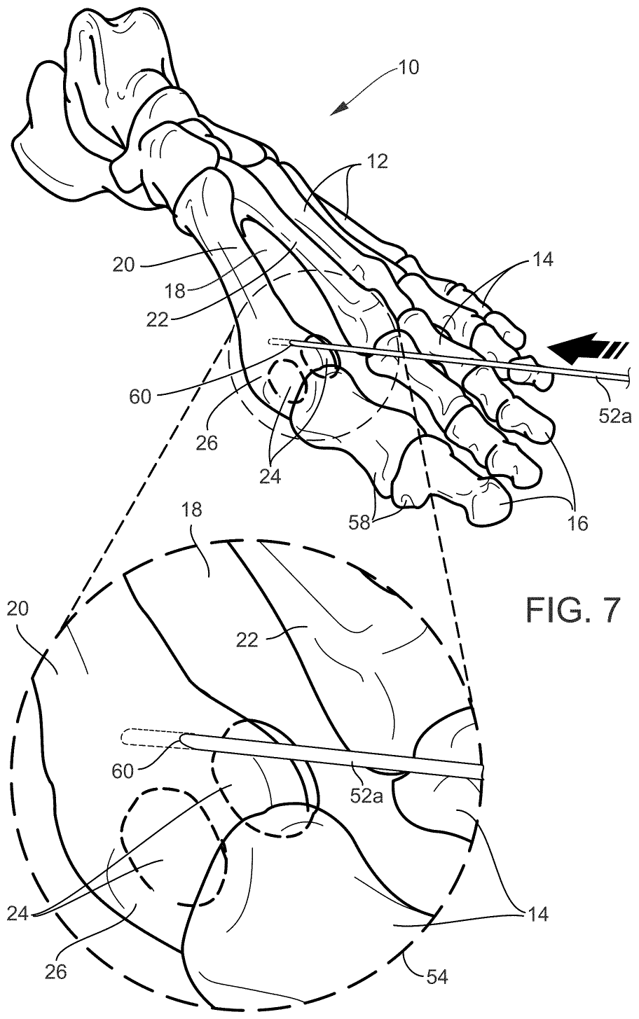

For a better understanding of the contemplated utilization of the depicted surgical tool 30a according to the invention, an example minimum incision bunion correction surgery is depicted in FIGS. 7-10. The surgery shown and described in FIGS. 7-10 is performed with the objective of achieving the bunion correction discussed supra with respect to FIGS. 4A-C.

Referring to FIG. 7, the depicted foot 10 is shown with a magnification 54 of the skeletal features near the first metatarsal head 26. A surgeon employing a minimum incision technique will make a small skin incision (not shown) near the first metatarsal head 26. The positioning guide that is in this example the guide wire 52a is then inserted into the skin incision and driven with a wire insertion apparatus (not shown) at an approximately 45 degree angle from the dorsal aspect of the hallux 58 into the first metatarsal head 20 as shown in FIG. 7. The guide wire 52a is typically a length of a 0.45 mm diameter k-wire that is sufficiently long enough to extend the entire length of the guide hole 48a of the surgical tool 30a and to also allow an additional remaining length for further guide manipulation. Although a wire positioning guide is shown and described in this example, it will be appreciated that other types of positioning guides such as long pins, rods, stiffened strings, rods, dowels, or any structure allowing for tool alignment can be similarly used within the contemplated scope of the invention.

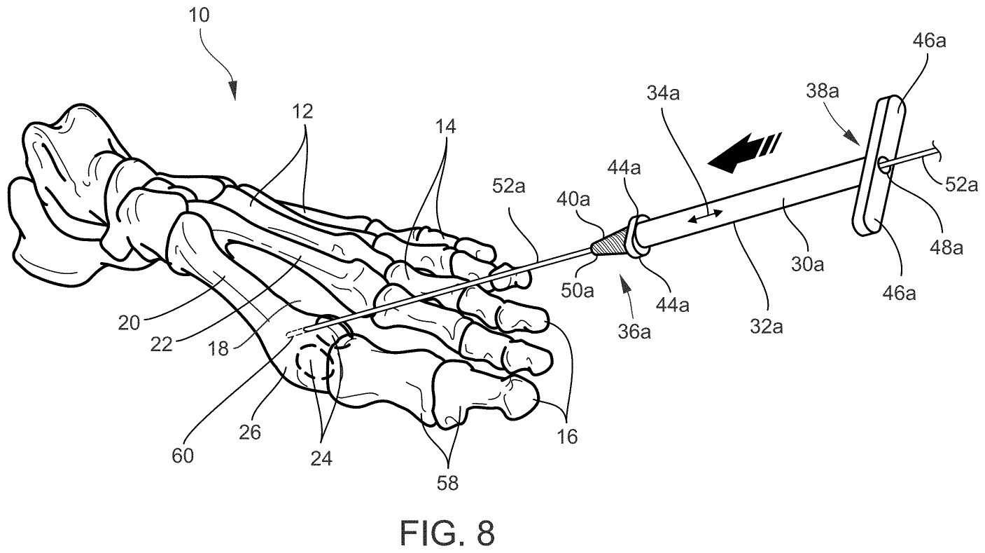

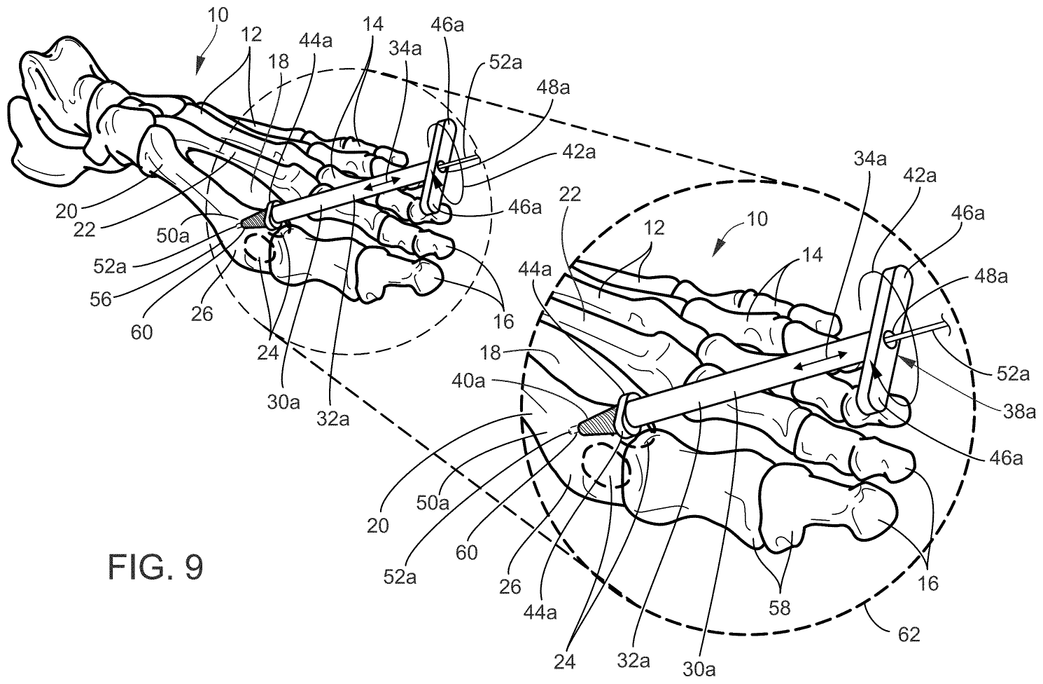

Comparing FIG. 7 with FIG. 8, the positioning guide or guide wire 52a is inserted through the guide hole 48a. Now comparing FIG. 8 with FIG. 9 and its magnification 62, as the surgical tool 36a is moved along the guide wire 52a toward the foot 10, the shaping and reduced size of the tool stop 44a enables the surgeon to maneuver or “tease” the surgical tool 36a into the incision and around surrounding skin and muscle (not shown). As depicted in FIG. 9, the point of wire insertion into the first metatarsal head 26 defines a boring position 60 for the surgical tool 30a and threads 40a. The clockwise threads 40a are positioned to allow the surgical tool 30a to bore into and engage the first metatarsal head when rotated in a right, clockwise boring direction 42a around the main body axis 34a.

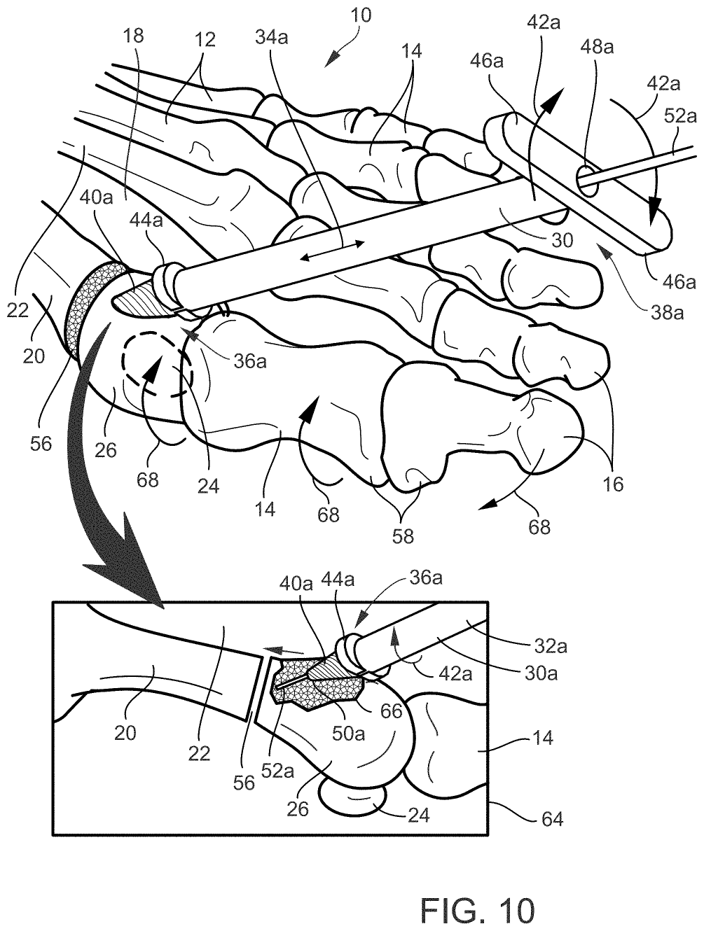

Referring to FIG. 10 and its inset view 64, the surgical tool 30a and its clockwise threads 40a, aligned in correct orientation by the guide wire 52a, continue to bore into the bone material of the first metatarsal head 26 as the surgeon applies pressure from the handling end 38a while rotating the surgical tool 30a in the clockwise boring direction 42a using handles 46a. As shown in the bone cutaway 66 of the inset view 64, the threads 40a create an increasingly tightened threaded engagement with the bone material of the first metatarsal head 26 as the surgical tool 30a continues to rotate in the clockwise boring direction 42a. Although the required amount of rotation may vary depending of several factors such as thread density and individual bone characteristics, at least three to four threads 40a may typically be required to be fully rotatably inserted for the tool 30a to stay securely seated into the metatarsal head 26. This tightening engagement continues until at least one leg of the tool stop 44a comes into contact with the first metatarsal head 26 to prevent further tool rotation. Once the tool stop 44a contacts bone material, the tool stop 44a is positioned to prevent the tool 30a from being further rotated around the main body axis 34a to prevent further boring.

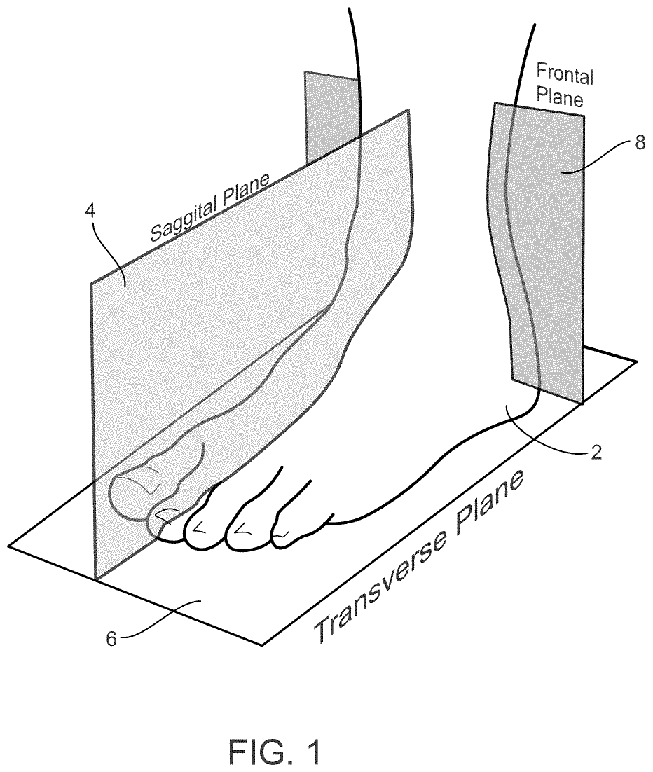

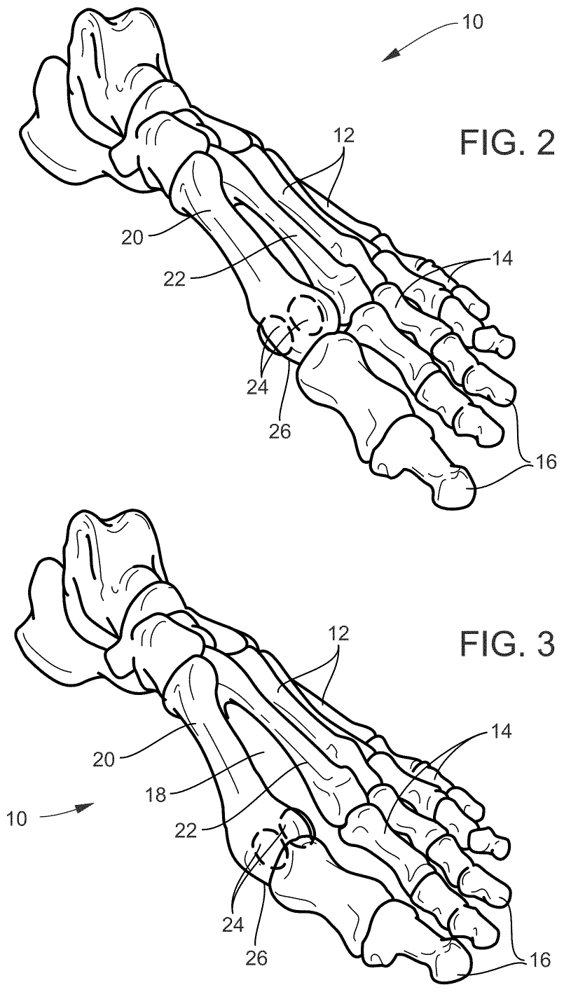

The surgeon employing this technique will next make a severing transverse cut or osteotomy 56 at the neck (diaphysial-metaphyseal junction) of the first metatarsal bone 20. Comparing FIG. 10 with FIG. 1, the osteotomy creates a free capital fragment that includes the first metatarsal head 26, which is pushed laterally for transverse plane correction. However, frontal plane correction is normally still necessary in order to attain proper cartilaginous alignment with the proximal phalanx of the hallux 58.

Once the free capital fragment/first metatarsal head 26 has been severed from the remainder for the first metatarsal bone 20 at the osteotomy 56, the frontal plane correction can be achieved by further rotating the surgical tool 30a in the right, clockwise boring direction 42a. The osteotomy 56 allows the free capital fragment/first metatarsal head 26 to move independently from the remainder of the first metatarsal bone 20. When the surgical tool 30a is further rotated in the right, clockwise boring direction 42a, the tool stop 44a prevents further boring by the tool 30a while providing rotational leverage. As a result, the tightened, clockwise engagement of the boring threads 40a with the capital fragment/first metatarsal head 26 causes bone rotation 68 of the metatarsal head 26 along with the clockwise rotation 42a of the surgical tool 30a. Although the tool stop 44a impedes further boring by the threads 40a, the clockwise rotation 42a of the surgical tool 30a nevertheless serves to maintain and tighten the clockwise engagement between the threads 40a and capital fragment/first metatarsal head 26, further securing the desired tool-bone engagement.

As the surgical tool 30a is rotated in the right, clockwise direction 42a, the resulting bone rotation 68 of the capital fragment/first metatarsal head 26 allows for the desired corrective bone repositioning along the frontal plane as depicted in FIG. 4C. The frontal plane correction also allows the sesamoid bones 24 to rotate back into proper alignment. This causes the flexor hallucis longos tendon (not shown) to have a more straight line effect on the hallux 58 after the surgical correction. The right, clockwise rotation tightened, clockwise engagement of the boring threads 40a with the capital fragment/first metatarsal head 26 also allows for fixed linear movement of the capital fragment, by the surgical tool 30a in any of the frontal, saggital, or transverse planes. Thus, the surgical tool 30a allows for substantial manipulation, movement, and rotation of bone while the tool 30a and bone are in tightened engagement.

Referring again to FIGS. 7-10, once frontal plane correction and any other manipulation of the capital fragment/first metatarsal head 26 has been completed, the guide wire 52a can be advanced across the osteotomy 56 to hold the corrected relative positioning until the surgical tool 30a is removed and permanent fixation is inserted. Once the osteotomy 56 is adequately fixated in its corrected position, the guide wire 52a can be removed.

Although the invention has been shown and described for use in performing bunion correction surgery on the human foot, it is contemplated the invention can also be used for other types of surgery and/or in other parts of both human and animal bodies wherever tight engagement is desired for bone rotation or linear bone movement or displacement. Selection of a tool using either clockwise or counterclockwise threads will normally depend on the anticipated need for rotation. For most applications, it would be desirable to select a tool with threads matching the anticipated direction of rotational repositioning.

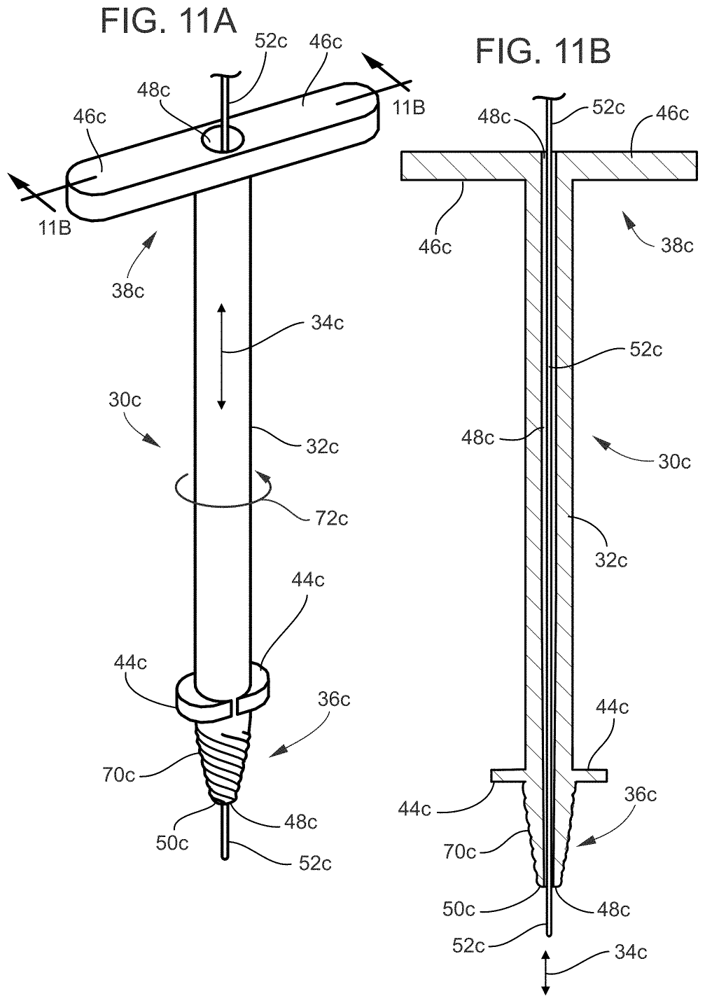

For example, FIG. 11A depicts a front, perspective view of a surgical tool 30c of the invention similar to the surgical tool 30a of FIGS. 5A and B. For comparison, a front cross sectional view of the surgical tool 30c taken along line 11B-11B of FIG. 11A is depicted in FIG. 11B. The surgical tool 30c of FIGS. 11A and B has a guide hole 48c for accommodating a guide wire 52c, an elongated tool body 32c and tool axis 34c with handles 46c at the handling end 38c and a tool stop 44e at the threaded end 36c. Conically shaped, counterclockwise boring threads 70c extend along the tool axis 34c below the tool stop 70c. The counter clockwise threads 70c allow the surgical tool 30c to be used for left, counterclockwise boring when rotated around the tool axis 34c in the boring direction 72c. This configuration would make the surgical tool 30e suitable for performing the depicted bunion correction surgery of FIGS. 7-10 on a right foot.

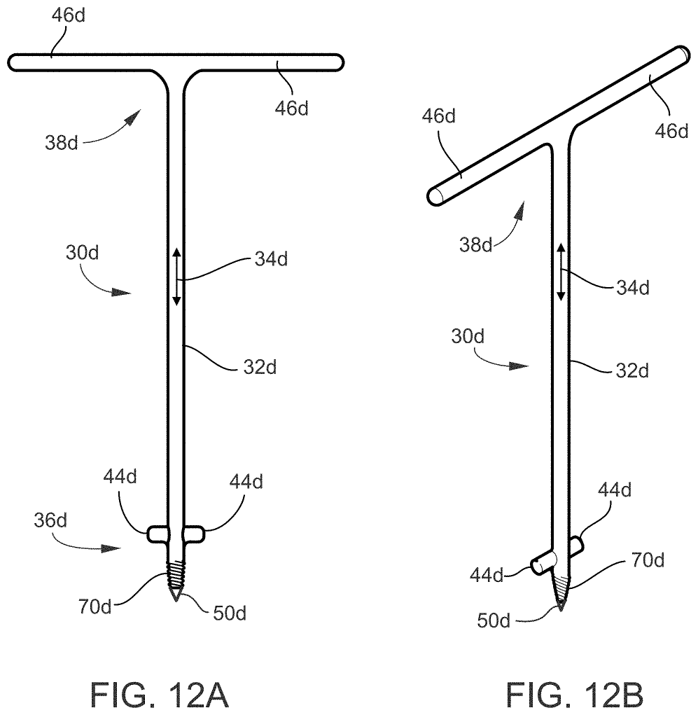

Although the invention has been shown and described with the use of a positioning guide such as the guide wire 52a in FIGS. 5A and B and 7-10, it will be appreciated that free-handed boring tool designs and surgical techniques are also contemplated within the intended scope of the invention, as are variations in tool dimensions and features. For example, FIGS. 12 A and B depict a surgical tool 30d of the invention lacking a guide hole and optimized for use without a guide wire. The tool body 32d has a reduced girth along tool axis 34d as do the handles 46d. Peg shaped legs form the tool stop 44d at the threaded end 36d. Since the surgical tool 30d lacks a guide hole, the conically shaped, counterclockwise boring threads 70d end at a pointed tip 50d similar to that depicted in FIG. 6B. In the embodiments depicted in FIGS. 12A and B, the pointed tip 50d could be advantageous for enhanced bone penetration when conducting a free-handed rotation of the surgical tool 30d.

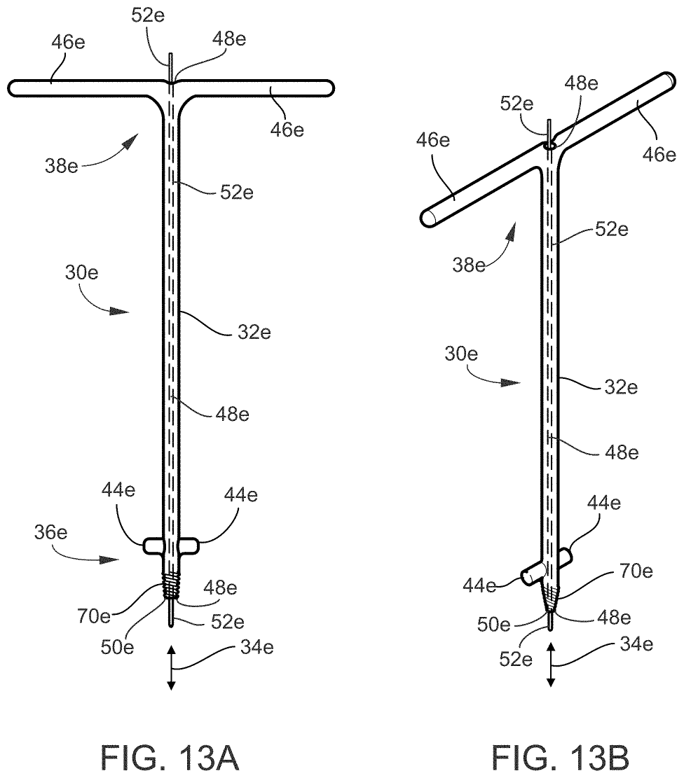

It will be further appreciated that some contemplated embodiments may still utilize a guide hole and positioning guide even if the tool body has a reduced girth. For example, FIGS. 13A and B depicts a surgical tool 30e of the invention having a guide hole 48e along the tool axis 34e for accommodating the guide wire 52e notwithstanding a reduced girth tool body 32e. The conically shaped, counterclockwise boring threads 70e extend along the tool axis 34c below the tool stop 70c and, despite the reduced girth of the tool body 32e, allow the guide hole 48e to open at the tip 50c to eliminate the need for free-handed positioning and rotation of the surgical tool 30e.

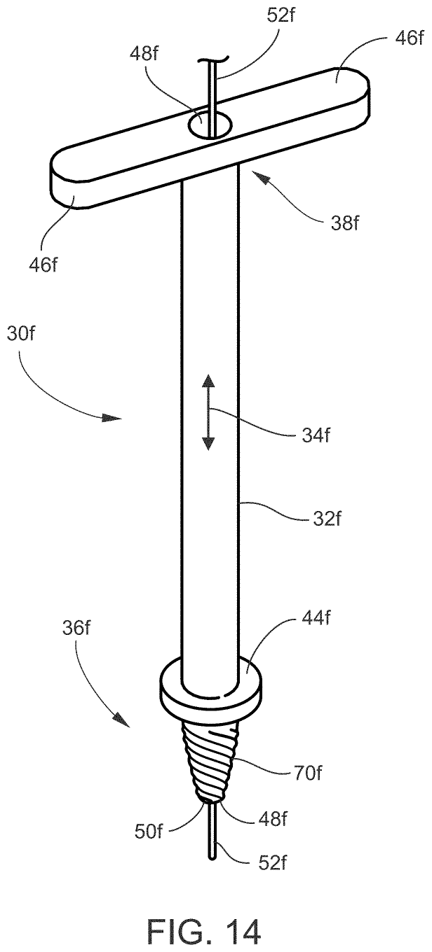

Other variations in the tool stop are also possible within the contemplated scope of the invention. For example, FIG. 14 depicts a surgical tool 30f having a guide hole 48f for accommodating a guide wire 52f, an elongated tool body 32f and tool axis 34f with handles 46f at the handling end 38f. However, the tool stop 44f at the threaded end 36f comprises a flange 44f extending completely around the main body axis 34f.

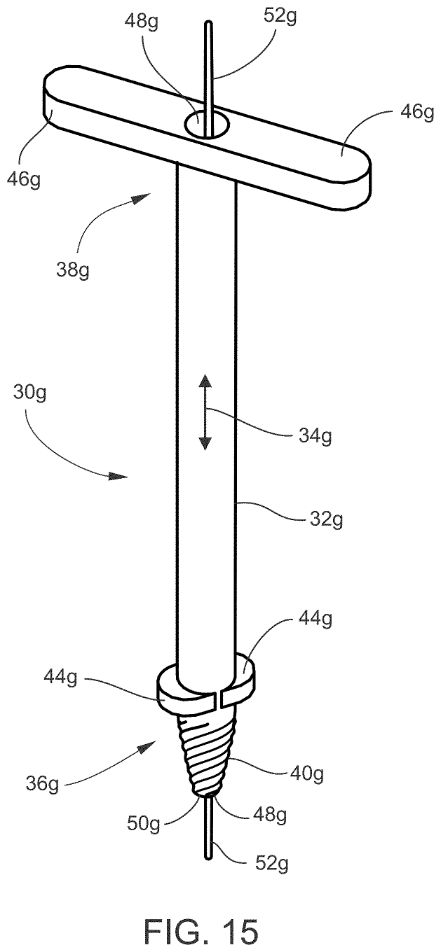

Although the invention has been shown and described using a tool in which the handles, tool body, and legs all lie substantially within a shared plane, it will be appreciated the invention can also include tools where such elements occupy different planes. For example, FIG. 15 depicts a surgical tool 30g of the invention having handles 46g, a tool body 32g, and tool stop 44g where the two handles 46g extend outwardly from the tool body 34g at the handling end 38g along a plane that is approximately 90 degrees from the plane in which the two legs of the tool stop 44g extend outwardly from the threaded end 44g. Since the handles 46g and tool stop 44g each share a different plane with the tool body 32g, the surgical tool 30g will not lie flat on a flattened surface. Such configurations could be desirable in applications or surgical environments where it is considered more difficult to grasp or retrieve the surgical tool 30g from an instrument table.

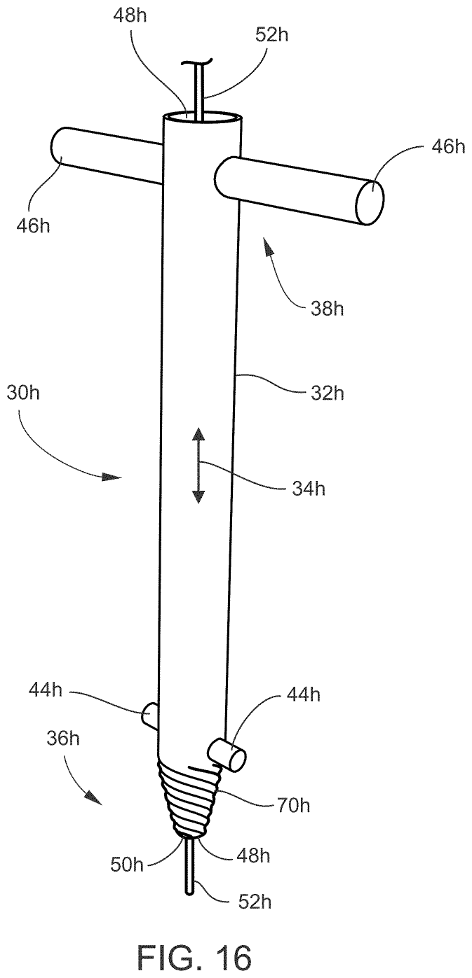

Other variations in handle, tool stop, or tool body shape and sizing are also possible and within the intended scope of the invention. For example, FIG. 16 depicts a surgical tool 30h of the invention having peg-shaped guide holes 46h extending from the handling end 38b of a tool body 32b having an enlarged girth and guide hole 48h. The legs of the tool stop 44h extending from the threaded end 36h are also peg-shaped and extend outwardly along the same plane as the tool body 32h and handles 46h. The enlarged girth of the tool body 32h would allow the counterclockwise boring threads 70h to create a larger threaded engagement with the first metatarsal head 26 during a right foot bunion correction surgery similar to that shown and described in FIGS. 7-10.

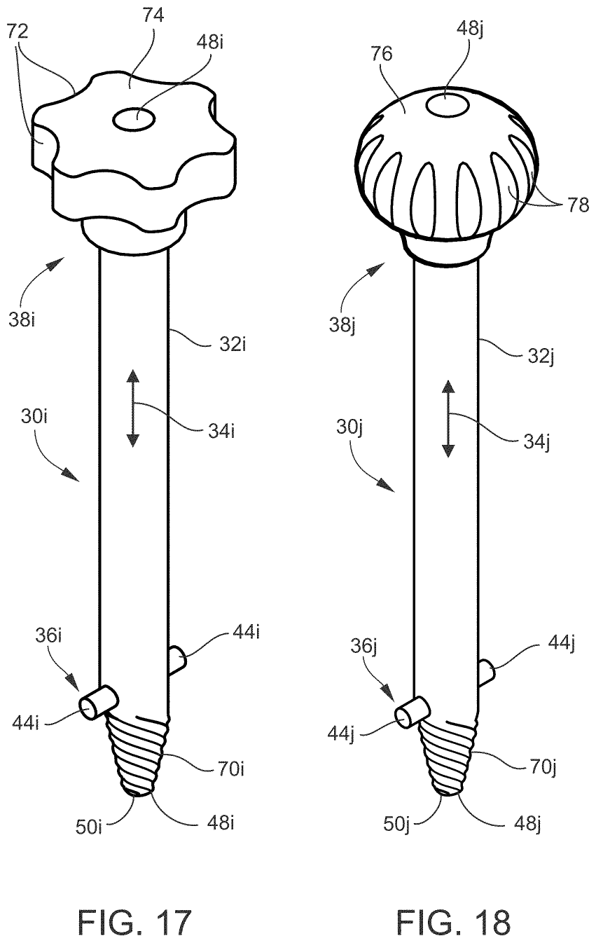

It will be further appreciated that the invention can incorporate other various alternative configurations of handles and/or knobs at the handling end of the surgical tool within the intended scope of the invention. For example, FIG. 17 depicts a surgical tool 301 of the invention having finger grooves 72 positioned around the perimeter of a grooved handle 74 at the handling end 381. In this configuration, the guide hole 481 of the surgical tool 301 extends along the main body axis 34i and through the grooved handle 74 to the tip 501 of the counterclockwise threads 701. The finger grooves 72 facilitate single-handed manipulation of the surgical tool 30i as the surgeon rotates the tool 301 and its counterclockwise threads 701 around its main body axis 34i.

One variation of the grooved handle surgical tool 301 of FIG. 17 is the surgical tool 30j depicted in FIG. 18 having a domed handle 76 that includes dome notches 78 around its outer circumference. The surgical tool 30j also includes a guide hole 48j extending through the top of the domed handle 76 and along the main body axis 34j through the tip 50j of the counterclockwise threads 70j. The finger grooves 72 and domes handle 76 also facilitate single-handed manipulation of the surgical tool 30j as the surgeon rotates the tool 30j and its counterclockwise threads 70j around its main body axis 34j.

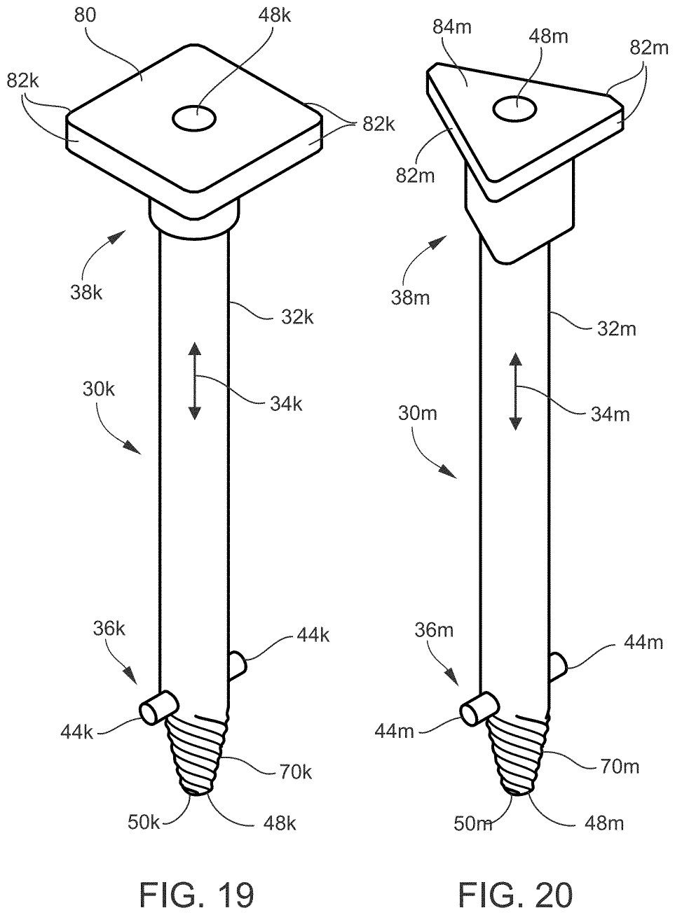

The surgical tools 301 and 30j of FIGS. 17 and 18 are potentially advantageous in preserving the single-handed utility of the invention while reducing the outward extending dimensions from the tool bodies 32i and 32j. Other configurations having similar advantages are also possible within the contemplated invention scope. For example, FIG. 19 depicts a surgical tool 30k of the invention having a square handle 80 with four straight edges 82k to enhance single-handed manipulation during surgery while still allowing for a guide hole 48k to extend into the tool body 32k while the dimensions of the surgical tool 30k extending outward from the tool body 32k are similarly reduced.

A slight variation is depicted in the surgical tool 30w of FIG. 20 having a triangle handle 84m with three straight edges 82m. The three straight edges 82w also allow for single-handed manipulation and include a guide hole 48w extending into the tool body 32m and opening through the tip 50m of the counterclockwise threads 70w at the threaded end 36m of the tool 30m.

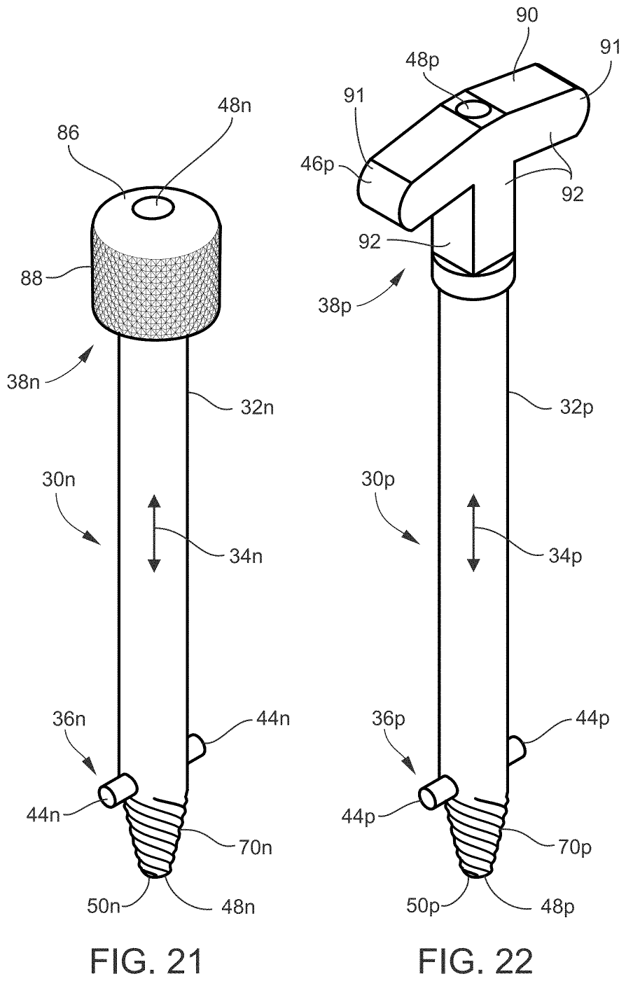

A further variation is depicted in the surgical tool 30n of FIG. 21 having a circular handle 86 with a gnarled outer surface 88. The gnarled outer surface 88 is roughened to allow enhanced tool manipulation despite the rolled shape of the outer surface 88. The circular handle 86 also includes a guide hole 48n extending into the tool body 32n and opening through the tip 50n of the counterclockwise threads 70n at the threaded end 36n of the tool 30n.

It is also possible to reduce surgical tool dimensions using a two-winged handle configuration and/or to configure a surgical tool of the invention to allow additional manipulation with an external tool or apparatus. For example, FIG. 22 depicts a surgical tool 30p of the invention having a proud T-handle 90 positioned at the handling end 38p and having two wings 91 extending outwardly from the tool body 32p substantially along a plane shared with the tool body 32p and legs of the tool stop 44p. The proud T-handle 90 also includes flattened tool engagement surfaces 92 that enable machine or external tool engagement with the surgical tool 30p for additional tool manipulation. The proud T-handle 90 also includes a guide hole 48p extending into the tool body 32p and opening through the tip 50p of the counterclockwise threads 70p at the threaded end 36p of the tool 30p.

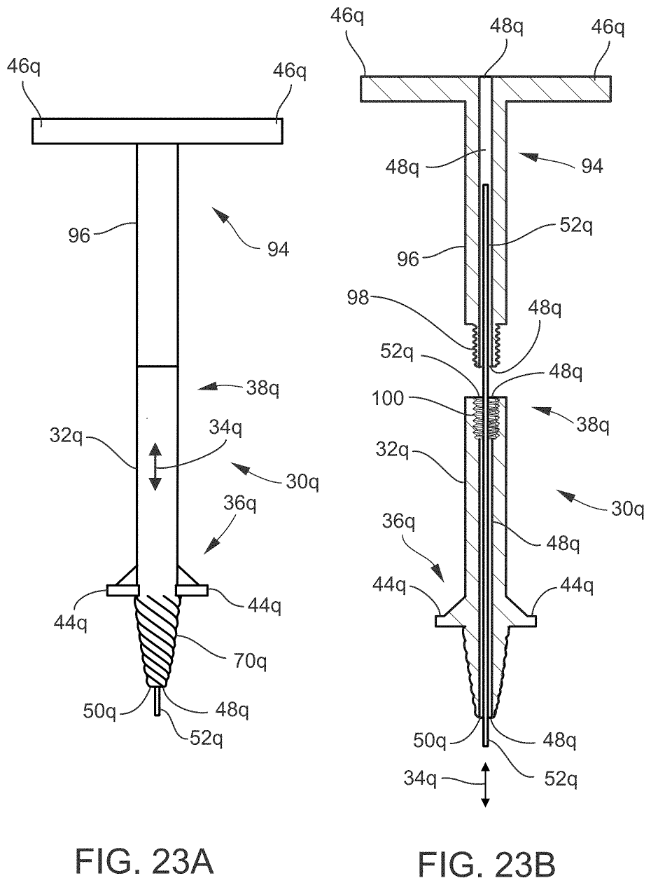

It is further contemplated that some embodiments can incorporate extension structures or multiple component body constructions, especially where it may be advantageous to conceal all or part of the positioning guide or to increase distance between the boring position and handles or other manipulation structures. For example, FIG. 23A depicts a front view of a surgical tool 30q of the invention having a detachable extension 94 attached to the handling end 38q of the tool body 32q. An exploded cross sectional view of the surgical tool 30q is depicted in FIG. 23B. As best understood by comparing FIG. 23A with FIG. 23B, the extension 94 includes handles 46q and an extension body 96 having male extension threads 98. The male extension threads 98 are positioned to screw into female handling threads 100 of the guide hole 48q at the handling end 38q of the tool body 32q. When the extension 94 has been fully screwed into position at the handling end 38q of the tool body 32q, the extension body 96 extends along the main body axis 34q. In this position, the guide hole 48q also extends along the main body axis 34q fully through the extension body 96, opening at the top of the extension 94 between the handles 46q.

As depicted in FIGS. 23A and B, a length of guide wire 52q has been selected and extends entirely though the tool body 32q, with a substantial length of exposed wire extending past the handling end 38q and out of the guide hole 52q. This would typically be considered advantageous after driving the guide wire 52q into bone and as the tool body 32q is positioned over the guide wire 52q itself. However, it would normally be considered problematic during the rotational boring of the counterclockwise threads 70q into the bone as the wire 52q could be considered an obstruction to the surgeon as the tool 30q is rotated.

Further comparing FIGS. 23A and B, attaching the extension 94 to the handling end 38q of the tool body 32q and over the remaining exposed length of guide wire 52q enables the remaining exposed length of wire 52q to be fully contained within the combined guide hole 48q of the tool body 32q and extension body 96. In the specific embodiment of FIGS. 23 A and B, the male extension threads 98 of the extension 94 and female handling threads 100 of the tool body 32a would normally be counterclockwise tightening to allow the engagement between the threads 98 and 100 to tighten as the surgeon rotates the surgical tool 30q around the main body axis 34q, boring into bone with the counterclockwise threads 70q. However, it will be appreciated that in other contemplated embodiments, similar male extension threads and female handling threads would be positioned clockwise tightening to allow for tightening engagement for tools having clockwise positioned threads.

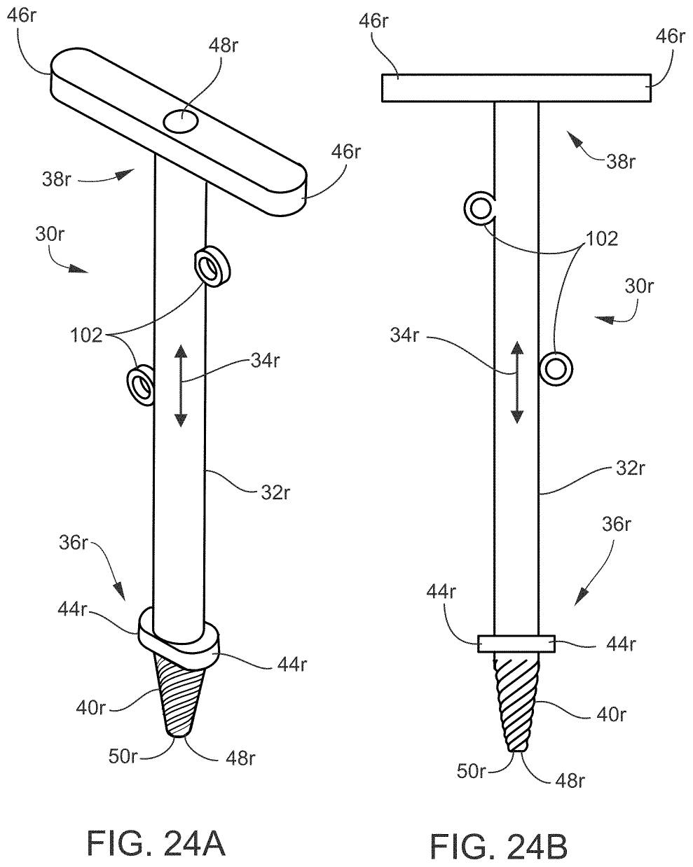

It will be further appreciated that in some contemplated embodiments, additional accessories can be added at various locations on the surgical tool for specific surgical purposes within the intended scope of the invention. For example, FIGS. 24A and B depict a surgical tool 30r of the invention having wire loops 102 positioned at locations along the outside surface of the tool body 32r. In the embodiment depicted in FIGS. 24A and B, one loop 102 is positioned at a location closer to the handling end 38r of the tool 30r and the other loop 102 is positioned closer to the threaded end 36r. In this configuration, the loops 102 extend outwardly in opposite directions from the tool body 32r along a plane shared by the tool body 32r, handles 46r, and legs of the tool stop 44r. However, it will be appreciated that other number of loops and loop positioning would also be within the contemplated scope of the invention.

As depicted in FIGS. 24A and B, the loops 102 would be particularly useful in performing a surgery such as the bunion correction surgery shown and described in FIGS. 7-10, with the loops 102 allowing for the direction of additional wires (not shown) used to hold the severed capital fragment/metatarsal head 26 in position relative the remainder of the first metatarsal bone 20 after the creation of the osteotomy 56 and after the guide wire 52a and tool have been used to complete bone repositioning. Thus the loops would enable the additional wires to provide temporary fixation of the correction capital fragment/metatarsal head 26.

It will be further appreciated that additional tools and techniques can also be used in performing bone surgery techniques within the contemplated scope of the invention. For example, in some contemplated surgical methods a drill (not shown) can be used over the guide wire prior to the insertion of the tool. In such embodiments, a soft tissue protector (not shown) would be used.

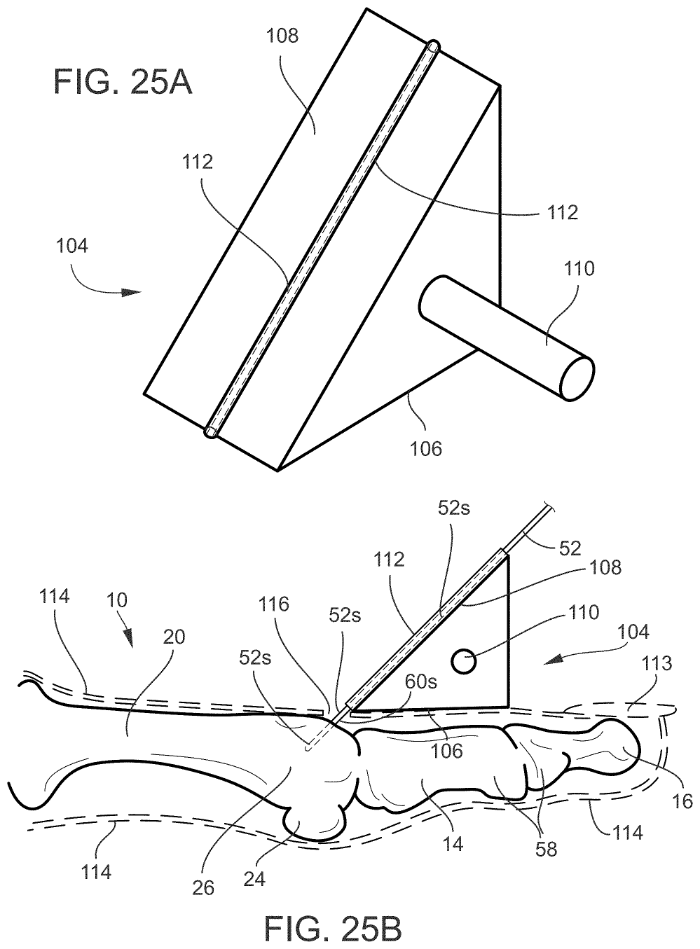

Referring now to FIG. 25A, another contemplated surgical technique of the invention would involve the use of an angulation tool 104 for alignment of guide wire. The angulation tool 104 includes a flattened bottom surface 106, angulated top surface 108, and side handle 110. The angulated top surface 108 is angled at 45 degrees from the flattened bottom surface 106 and includes a cannulated wire tube 112 extending on top of and along the length of the angulated top surface 108. The wire tube 112 is dimensioned to accommodate a length of k-wire and allows for positioning of the k-wire at 45 degrees to the flattened bottom surface 106.

FIG. 25B depicts a surgical technique utilizing the angulation tool 104 of FIG. 25A that is a modification of the bunion correction surgical technique depicted in FIGS. 7-10 for a left foot 10. Referring to FIG. 25B, a side view is depicted of a big toe nail 113 with skin and non-bone tissue 114 surrounding the first metatarsal bone 20, sesamoid bone 24, and hallux 58. A small incision 116 is made near the first metatarsal head 26. The angulation tool 104 is then positioned with its flattened bottom surface 106 placed over the hallux 58 so the wire tube 112 is oriented directly at 45 degrees with respect to the hallux 58 and first metatarsal head 26 as depicted.

Once the angulation tool 104 is in this position as shown in FIG. 25B, the guide wire 52s, typically a length of k-wire, is fed into the wire tube 112 and then guided into and through the incision 116 until it is proximate or in contact with the, first metatarsal head 26 at the boring position 60s and at the correct 45 degree angle provided by the angulation tool 104. A k-wire driver (not shown) is then used, while the guide wire 52s remains positioned by angulation tool 104, to drive the end of the guide wire 52s into the first metatarsal head 26 as depicted. The angulation tool 104 can then be removed, with the guide wire 52s remaining in position, and a tool of the invention such as the surgical tool 30a of FIGS. 5A and B then used to complete the repositioning of the metatarsal head 26.

Although the surgical technique depicted in FIGS. 25A and B depict the use of a angulation tool for optimally positioning a guide wire at 45 degrees from the hallux 58 and first metatarsal head 26, it will be appreciated that is some contemplated embodiments of the described surgical method and angulation tool, the preferred optimal angle of wire insertion may be predetermined to be 25-35 degrees or another degree of angulation with an appropriately angulated tool employed within the anticipated scope of the invention.

Those skilled in the art will realize that this invention is capable of embodiments different from those shown and described. It will be appreciated that the detail of the structure of the disclosed apparatuses and methodologies can be changed in various ways without departing from the invention itself. Accordingly, the drawings and detailed description are to be regarded as including such equivalents as do not depart from the spirit and scope of the invention.

1. A surgical tool for engaging and manipulating a bone comprising:
a tool body having a main body axis, a threaded end, and a handling end;
said threaded end having threads and a tool stop;
said handling end being positioned to allow a user to grasp, manipulate, and rotate said tool body around said main body axis;
said threads being positioned to allow said surgical tool to bore into and engage the bone when said tool body is rotated in a boring direction around said main body axis, said threads allowing for tightening engagement with the bone as said tool body is rotated in the boring direction around said main body axis;
said tool stop being positioned to prevent said surgical tool from being further rotated around said main body axis and to prevent further boring into the bone when said tool stop contacts the bone; and
said surgical tool allowing for manipulation, movement, and rotation of the bone with said surgical tool when said tool is in tightened engagement with the bone.

2. The surgical tool of claim 1 wherein said threads are positioned to bore into and engage the bone by said tool body being rotated around said main body axis in a right, clockwise direction.

3. The surgical tool of claim 1 wherein said threads are positioned to bore into and engage the bone by said tool body being rotated around said main body axis in a left, counterclockwise direction.

4. The surgical tool of claim 1 further comprising:
said tool stop having at, least one leg extending outwardly from said tool body;
said handling end having at least one handle extending outwardly from said tool body; and
said at least one leg of said tool stop and said at least one handle of said handling end lying substantially within a plane shared with said main body axis wherein said surgical tool remains substantially flat when placed on a flat surface.

5. The surgical tool of claim 1 further comprising:
said tool stop having two legs extending outwardly from said tool body;
said handling end having two handles extending outwardly from said tool body; and
both of said legs of said tool stop and both of said handles of said handling end lying substantially within a plane shared with said main body axis wherein said surgical tool remains substantially flat when placed on a flat surface.

6. The surgical tool of claim 1 wherein said tool stop comprises a flange.

7. The surgical tool of claim 1 wherein said tool stop comprises at least one tabular leg.

8. The surgical tool of claim 1 wherein said tool stop comprises at least one peg leg.

9. The surgical tool of claim 1 wherein said tool stop extends completely around said main body axis.

10. The surgical tool of claim 1 further comprising:
said tool body having handling threads positioned at said handling end;
an extension, said extension having an extension axis and extension threads;
said extension threads of said extension being threadably connected to said handling threads of said tool body wherein said extension axis of said extension is about in line with said main body axis of said tool body; and
said extension threads of said extension being positioned to tighten against said handling threads of said tool body when said extension and said tool body are rotated in the boring direction around said main body axis and said extension axis.

11. The surgical tool of claim 1 wherein said handling end is positioned for connection to an external drill for rotation of said surgical tool around said main body axis.

12. The surgical tool of claim 1 further comprising a knob at said handling end to allow for rotation of said surgical tool around said main body axis.

13. The surgical tool of claim 1 further comprising a grooved handle at said handling end to allow for rotation of said surgical tool around said main body axis.

14. The surgical tool of claim 1 further comprising a domed handle at said handling end to allow for rotation of said surgical tool around said main body axis.

15. The surgical tool of claim 1 further comprising a square handle at said handling end to allow for rotation of said surgical tool around said main body axis.

16. The surgical tool of claim 1 further comprising a triangle handle at said handling end to allow for rotation of said surgical tool around said main body axis.

17. The surgical tool of claim 1 further comprising a circle handle at said handling end to allow for rotation of said surgical, tool around said main body axis.

18. The surgical tool of claim 1 further comprising a proud T handle at said handling end to allow for rotation of said surgical tool around said main body axis.

19. The surgical tool of claim 1 further comprising an engagement surface for attachment of an external tool for rotating said tool body around said main body axis.

20. The surgical tool of claim 1 further comprising loops located at positions along said tool body to allow for the direction of additional wires to provide temporary fixation of bone material.

21. A surgical tool for engaging and manipulating a bone comprising:
a tool body having a main body axis, a threaded end, a handling end, and a guide hole extending through said tool body substantially along said main body axis;
said threaded end having threads and a tool stop;
said handling end being positioned to allow a user to grasp, manipulate, and rotate said tool body around said main body axis;
said guide hole allowing a positioning guide to extend at least partially through said tool body and into a bone at a bone boring position to align and guide said threads of said surgical tool to the bone boring position;
said threads being positioned to allow said surgical tool to bore at the bone boring position into and engage the bone when said tool body is rotated in a boring direction around said main body axis, said threads allowing for tightening engagement with the bone as said tool body is rotated in the boring direction around said main body axis;
said tool stop being positioned to prevent said surgical tool from being further rotated around said main body axis and to prevent further boring into the bone when said tool stop contacts the bone; and
said surgical tool allowing for manipulation, movement, and rotation of the bone with said surgical tool when said tool is in tightened engagement with the bone.

22. The surgical tool of claim 21 wherein said threads are positioned to bore into and engage the bone by said tool body being rotated around said main body axis in a right, clockwise direction.

23. The surgical tool of claim 21 wherein said threads are positioned to bore into and engage the bone by said tool body being rotated around said main body axis in a left, counterclockwise direction.

24. The surgical tool of claim 21 wherein the positioning guide is a guide wire.

25. The surgical tool of claim 21 wherein the positioning guide is a guide wire pre-embedded in the bone at the bone boring position.

26. The surgical tool of claim 21 wherein the positioning guide is 0.45 mm diameter k-wire.

27. The surgical tool of claim 21 further comprising:
said tool stop having at least one leg extending outwardly from said tool body;
said handling end having at least one handle extending outwardly from said tool body; and
said at least one leg of said tool stop and said at least one handle of said handling end lying substantially within a plane shared with said main body axis wherein said surgical tool remains substantially flat when placed on a flat surface.

28. The surgical tool of claim 21 further comprising:
said tool stop having two legs extending outwardly from said tool body;
said handling end having two handles extending outwardly from said tool body; and
both of said legs of said tool stop and both of said handles of said handling end lying substantially within a plane shared with said main body axis wherein said surgical tool remains substantially flat when placed on a flat surface.

29. The surgical tool of claim 21 wherein said tool stop comprises a flange.

30. The surgical tool of claim 21 wherein said tool stop comprises at least one tabular leg.

31. The surgical tool of claim 21 wherein said tool stop comprises at least one peg leg.

32. The surgical tool of claim 21 wherein said tool stop extends completely around said main body axis.

33. The surgical tool of claim 21 further comprising:
said tool body having handling threads positioned at said handling end;
an extension, said extension having an extension axis, an extension hole extending substantially along said extension axis, and extension threads;
said extension threads of said extension being threadably connected to said handling threads of said tool body wherein said extension axis of said extension is about in line with said main body axis of said tool body and said guide hole is about in line with said extension hole;
said guide hole and said extension hole being positioned to allow said positioning guide to extend at least partially through said tool body and said extension; and
said extension threads of said extension being positioned to tighten against said handling threads of said tool body when said extension and said tool body are rotated in the boring direction around said main body axis and said extension axis.

34. The surgical tool of claim 21 wherein said handling end is positioned for connection to an external drill for rotation of said surgical tool around said main body axis.

35. The surgical tool of claim 21 further comprising a knob at said handling end to allow for rotation of said surgical tool around said main body axis.

36. The surgical tool of claim 21 further comprising a grooved handle at said handling end to allow for rotation of said surgical tool around said main body axis.

37. The surgical tool of claim 21 further comprising a domed handle at said handling end to allow for rotation of said surgical tool around said main body axis.

38. The surgical tool of claim 21 further comprising a square handle at said handling end to allow for rotation of said surgical tool around said main body axis.

39. The surgical tool of claim 21 further comprising a triangle handle at said handling end to allow for rotation of said surgical tool around said main body axis.

40. The surgical tool of claim 21 further comprising a circle handle at said handling end to allow for rotation of said surgical tool around said main body axis.

41. The surgical tool of claim 21 further comprising a proud T handle at said handling end to allow for rotation of said surgical tool around said main body axis.

42. The surgical tool of claim 21 further comprising an engagement surface for attachment of an external tool for rotating said tool body around said main body axis.

43. The surgical tool of claim 21 further comprising an engagement surface for attachment of an external tool for rotating said tool body around said main body axis.

44. The surgical tool of claim 21 further comprising loops located at positions along said tool body to allow for the direction of additional wires to provide temporary fixation of bone material.

45. A surgical tool for performing bunion surgery on a left foot comprising:
a tool body having a main body axis, a threaded end, a handling end, and a guide hole extending through said tool body substantially along said main body axis;
said threaded end having threads and a tool stop;
said handling end being positioned to allow a user to grasp, manipulate, and rotate said tool body around said main body axis;
said guide hole allowing a positioning guide to extend at least partially through said tool body and into the first metatarsal head of the left foot at a bone boring position to align and guide said threads of said surgical tool to the bone boring position;
said threads being positioned to allow said surgical tool to bore at the bone boring position into and engage the first metatarsal head when said tool body is rotated in a right, clockwise boring direction around said main body axis, said threads allowing for tightening engagement with the first metatarsal head as said tool body is rotated in the right, clockwise boring direction around said main body axis;
said tool stop being positioned to prevent said surgical tool from being further rotated around said main body axis in the right, clockwise direction and to prevent further boring into the first metatarsal head when said tool stop contacts bone; and
said surgical tool allowing for manipulation and movement of the first metatarsal head in the frontal, saggital, and transverse planes of the left foot, and right, clockwise rotation of the first metatarsal head, when said surgical tool is in tightened engagement with the first metatarsal head.

46. The surgical tool of claim 45 wherein the positioning guide is a guide wire.

47. The surgical tool of claim 45 wherein the positioning guide is a guide wire pre-embedded in the bone at the bone boring position.

48. The surgical tool of claim 45 wherein the positioning guide is 0.45 mm diameter k-wire.

49. The surgical tool of claim 45 further comprising:
said tool stop having at least one leg extending outwardly from said tool body;
said handling end having at least one handle extending outwardly from said tool body; and
said at least one leg of said tool stop and said at least one handle of said handling end lying substantially within a plane shared with said main body axis wherein said surgical tool remains substantially flat when placed on a flat surface.

50. The surgical tool of claim 45 further comprising:
said tool stop having two legs extending outwardly from said tool body;
said handling end having two handles extending outwardly from said tool body; and
both of said legs of said tool stop and both of said handles of said handling end lying substantially within a plane shared with said main body axis wherein said surgical tool remains substantially flat when placed on a flat surface.

51. The surgical tool of claim 45 wherein said tool stop comprises a flange.

52. The surgical tool of claim 45 wherein said tool stop comprises at least one tabular leg.

53. The surgical tool of claim 45 wherein said tool stop comprises at least one peg leg.

54. The surgical tool of claim 45 wherein said tool stop extends completely around said main body axis.

55. The surgical tool of claim 45 further comprising:
said tool body having handling threads positioned at said handling end;
an extension, said extension having an extension axis, an extension hole extending substantially along said extension axis, and extension threads;
said extension threads of said extension being threadably connected to said handling threads of said tool body wherein said extension axis of said extension is about in line with said main body axis of said tool body and said guide hole is about in line with said extension hole;
said guide hole and said extension hole being positioned to allow said positioning guide to extend at least partially through said tool body and said extension; and
said extension threads of said extension being positioned to tighten against said handling threads of said tool body when said extension and said tool body are rotated in the boring direction around said main body axis and said extension axis.

56. The surgical tool of claim 45 wherein said handling end is positioned for connection to an external drill for rotation of said surgical tool around said main body axis.

57. The surgical tool of claim 45 further comprising a knob at said handling end to allow for rotation of said surgical tool around said main body axis.

58. The surgical tool of claim 45 further comprising a grooved handle at said handling end to allow for rotation of said surgical tool around said main body axis.

59. The surgical tool of claim 45 further comprising a domed handle at said handling end to allow for rotation of said surgical tool around said main body axis.

60. The surgical tool of claim 45 further comprising a square handle at said handling end to allow for rotation of said surgical tool around said main body axis.

61. The surgical tool of claim 45 further comprising a triangle handle at said handling end to allow for rotation of said surgical tool around said main body axis.

62. The surgical tool of claim 45 further comprising a circle handle at said handling end to allow for rotation of said surgical tool around said main body axis.

63. The surgical tool of claim 45 further comprising a proud T handle at said handling end to allow for rotation of said surgical tool around said main body axis.

64. The surgical tool of claim 45 further comprising an engagement surface for attachment of an external tool for rotating said tool body around said main body axis.

65. The surgical tool of claim 45 further comprising an engagement surface for attachment of an external tool for rotating said tool body around said main body axis.

66. The surgical tool of claim 45 further comprising loops located at positions along said tool body to allow for the direction of additional wires to provide temporary fixation of bone material.

67. A surgical tool for performing bunion surgery on a right foot comprising:
a tool body having a main body axis, a threaded end, a handling end, and a guide hole extending through said tool body substantially along said main body axis;
said threaded end having threads and a tool stop;
said handling end being positioned to allow a user to grasp, manipulate, and rotate said tool body around said main body axis;
said guide hole allowing a positioning guide to extend at least partially through said tool body and into the first metatarsal head of the right foot at a bone boring position to align and guide said threads of said surgical tool to the bone boring position;
said threads being positioned to allow said surgical tool to bore at the bone boring position into and engage the first metatarsal head when said tool body is rotated in a left, counterclockwise boring direction around said main body axis, said threads allowing for tightening engagement with the first metatarsal head as said tool body is rotated in the left, counterclockwise boring direction around said main body axis;
said tool stop being positioned to prevent said surgical tool from being further rotated around said main body axis in the left, counterclockwise direction and to prevent further boring into the first metatarsal head when said tool stop contacts the bone; and
said surgical tool allowing for manipulation and movement of the first metatarsal head in the frontal, saggital, and transverse planes of the right foot, and left, counterclockwise rotation of the first metatarsal head, when said surgical tool is in tightened engagement with the first metatarsal head.

68. The surgical tool of claim 67 wherein the positioning guide is a guide wire.

69. The surgical tool of claim 67 wherein the positioning guide is a guide wire pre-embedded in the bone at the bone boring position.

70. The surgical tool of claim 67 wherein the positioning guide is 0.45 mm diameter k-wire.

71. The surgical tool of claim 67 further comprising:
said tool stop having at least one leg extending outwardly from said tool body;
said handling end having at least one handle extending outwardly from said tool body; and
said at least one leg of said tool stop and said at least one handle of said handling end lying substantially within a plane shared with said main body axis wherein said surgical tool remains substantially flat when placed on a flat surface.

72. The surgical tool of claim 67 further comprising:
said tool stop having two legs extending outwardly from said tool body;
said handling end having two handles extending outwardly from said tool body; and
both of said legs of said tool stop and both of said handles of said handling end lying substantially within a plane shared with said main body axis wherein said surgical tool remains substantially flat when placed on a flat surface.

73. The surgical tool of claim 67 wherein said tool stop comprises a flange.

74. The surgical tool of claim 67 wherein said tool stop comprises at least one tabular leg.

75. The surgical tool of claim 67 wherein said tool stop comprises at least one peg leg.

76. The surgical tool of claim 67 wherein said tool stop extends completely around said main body axis.

77. The surgical tool of claim 67 further comprising:
said tool body having handling threads positioned at said handling end;
an extension, said extension having an extension axis, an extension hole extending substantially along said extension axis, and extension threads;
said extension threads of said extension being threadably connected to said handling threads of said tool body wherein said extension axis of said extension is about in line with said main body axis of said tool body and said guide hole is about in line with said extension hole;
said guide hole and said extension hole being positioned to allow said positioning guide to extend at least partially through said tool body and said extension; and
said extension threads of said extension being positioned to tighten against said handling threads of said tool body when said extension and said tool body are rotated, in the boring direction around said main body axis and said extension axis.

78. The surgical tool of claim 67 wherein said handling end is positioned for connection to an external drill for rotation of said surgical tool around said main body axis.

79. The surgical tool of claim 67 further comprising a knob at said handling end to allow for rotation of said surgical tool around said main body axis.

80. The surgical tool of claim 67 further comprising a grooved handle at said handling end to allow for rotation of said surgical tool around said main body axis.

81. The surgical tool of claim 67 further comprising a domed handle at said handling end to allow for rotation of said surgical tool around said main body axis.

82. The surgical tool of claim 67 further comprising a square handle at said handling end to allow for rotation of said surgical tool around said main body axis.

83. The surgical tool of claim 67 further comprising a triangle handle at said handling end to allow for rotation of said surgical tool around said main body axis.

84. The surgical tool of claim 67 further comprising a circle handle at said handling end to allow for rotation of said surgical tool around said main body axis.

85. The surgical tool of claim 67 further comprising a proud T handle at said handling end to allow for rotation of said surgical tool around said main body axis.

86. The surgical tool of claim 67 further comprising an engagement surface for attachment of an external tool for rotating said tool body around said main body axis.

87. The surgical tool of claim 67 further comprising an engagement surface for attachment of an external tool for rotating said tool body around said main body axis.

88. The surgical tool of claim 67 further comprising loops located at positions along said tool body to allow for the direction of additional wires to provide temporary fixation of bone material.

89. A surgical method comprising:
providing a surgical tool having a tool body, the tool body having a main body axis, a threaded end, and a handling end, the threaded end having threads and a tool stop, the handling end being positioned to allow a user to grasp, manipulate, and rotate the tool body around the main body axis;
rotating the surgical tool in a boring direction around the main body axis to bore into and engage the bone with the threads, further tightening engagement with the bone as the surgical tool continues to rotate in the boring direction around the main body axis;
continuing to rotate the surgical tool until the tool stop contacts the bone to prevent the surgical tool from being further rotated around the main body axis and to prevent further boring into the bone; and
manipulating, moving, and rotating the bone with the surgical tool while the surgical tool is in tightened engagement with the bone.

90. The surgical method of claim 89 wherein the threads of the tool body are rotated around the main tool body axis in a right, clockwise direction to bore into and engage the bone.

91. The surgical method of claim 89 wherein the threads of the tool body are rotated around the main tool body axis in a left, counterclockwise direction to bore into and engage the bone.

92. The surgical method of claim 89 wherein an external drill is connected to the handling end of the tool body, the drill then being used to rotate the surgical tool around the main body axis.

93. The surgical method of claim 89 further comprising:
providing loops located at positions along the tool body; and
directing additional wires through said loops and using the additional wires to provide temporary fixation of bone material.

94. A surgical method comprising:
providing a surgical tool having a tool body, the tool body having a main body axis, a threaded end, a handling end, and a guide hole extending through the tool body substantially along the main body axis, the threaded end having threads and a tool stop, the handling end being positioned to allow a user to grasp, manipulate, and rotate the tool body around said main body axis;
providing a positioning guide and inserting the positioning guide into a bone at a bone boring position;
extending the positioning guide through the guide hole at least partially through the tool body;
aligning and guiding with the positioning guide the threads of the surgical tool to the bone boring position;
rotating the surgical tool in a boring direction around the main body axis to bore into and engage the bone with the threads at the bone boring position, the threads further tightening engagement with the bone as the tool body continues to rotate in the boring direction around the main body axis;
continuing to rotate the surgical tool until the tool stop contacts the bone to prevent the tool from being further rotated around the main body axis and to prevent further boring into the bone; and
manipulating, moving, and rotating the bone with the surgical tool while the tool is in tightened engagement with the bone.

95. The surgical method of claim 94 wherein the threads of the tool body are rotated around the main tool body axis in a right, clockwise direction to bore into and engage the bone.

96. The surgical method of claim 94 wherein the threads of the tool body are rotated around the main tool body axis in a left, counterclockwise direction to bore into and engage the bone.

97. The surgical method of claim 94 wherein the positioning guide is a guide wire.

98. The surgical method of claim 94 wherein the positioning guide is a guide wire pre-embedded in the bone at the bone boring position.

99. The surgical method of claim 94 wherein the positioning guide is 0.45 mm diameter k-wire.

100. The surgical method of claim 94 wherein an external drill is connected to the handling end of the tool body, the drill then being used to rotate the surgical tool around the main body axis.

101. The surgical method of claim 94 further comprising:
providing loops located at positions along the tool body; and
directing additional wires through said loops and using the additional wires to provide temporary fixation of bone material.

102. The surgical method of claim 94 further comprising:
providing an angulation tool guide having a positioning angle;
positioning the angulation tool with respect to the boring position; and
aligning the positioning guide with the angulation tool at the positioning angle and inserting the positioning guide into bone at the boring position.

103. A method of performing bunion surgery on a left foot comprising:
providing a surgical tool having a tool body, the tool body having a main body axis, a threaded end, a handling end, and a guide hole extending through the tool body substantially along the main body axis, the threaded end having threads and a tool stop, the handling end being positioned to allow a user to grasp, manipulate and rotate the tool body around the main body axis;
providing a positioning guide and inserting the positioning guide into the first metatarsal head of the left foot at a bone boring position;
extending the positioning guide through the guide hole at least partially through the tool body;
aligning and guiding with the positioning guide the threads of the surgical tool to the bone boring position;
rotating the surgical tool in a right, clockwise boring direction around the main body axis to bore into and engage the first metatarsal head with the threads at the bone boring position, the threads further tightening engagement with the first metatarsal head as the tool body continues to rotate in the right, clockwise boring direction around the main body axis;
continuing to rotate the surgical tool in the right, clockwise direction until the tool stop contacts the first metatarsal head to prevent the surgical tool from being further rotated in the right, clockwise direction around the main body axis and to prevent further boring into the first, metatarsal head; and
manipulating and moving the first metatarsal head in the frontal, saggital, and transverse planes of the left foot, and effecting right, clockwise rotation of the first metatarsal head with the surgical tool as the surgical tool is in tightened engagement with the first metatarsal head.

104. The method of performing bunion surgery of claim 103 wherein the positioning guide is a guide wire.

105. The method of performing bunion surgery of claim 103 wherein the positioning guide is a guide wire pre-embedded in the first metatarsal head at the bone boring position.

106. The method of performing bunion surgery of claim 103 wherein the positioning guide is 0.45 mm diameter k-wire.

107. The method of performing bunion surgery of claim 103 wherein an external drill is connected to the handling end of the tool body, the drill then being used to rotate the surgical tool around the main body axis.

108. The method of performing bunion surgery of claim 103 further comprising:
providing loops located at positions along the tool body; and
directing additional wires through said loops and using the additional wires to provide temporary fixation of bone material.

109. The method of performing bunion surgery of claim 103 further comprising:
providing an angulation tool guide having a positioning angle;
positioning the angulation tool with respect to the boring position; and
aligning the positioning guide with the angulation tool at the positioning angle and inserting the positioning guide into the first metatarsal head at the boring position.

110. A method of performing bunion surgery on a right foot comprising:
providing, a surgical tool having a tool body, the tool body having a main body axis, a threaded end, a handling end, and a guide hole extending through the tool body substantially along the main body axis, the threaded end having threads and a tool stop, the handling end being positioned to allow a user to grasp, manipulate, and rotate the tool body around the main body axis;
providing a positioning guide and inserting the positioning guide into the first metatarsal head of the right foot at a bone boring position;
extending the positioning guide through the guide hole at least partially through the tool body;
aligning and guiding with the positioning guide the threads of the surgical tool to the bone boring position;
rotating the surgical tool in a left, counterclockwise boring direction around the main body axis to bore into and engage the first metatarsal head with the threads at the bone boring position, the threads further tightening engagement with the first metatarsal head as the tool body continues to rotate in the left, counterclockwise boring direction around the main body axis;
continuing to rotate the surgical tool in the left, counterclockwise direction until the tool stop contacts the first metatarsal head to prevent the surgical tool from being further rotated in the left, counterclockwise direction around the main body axis and to prevent further boring into the first metatarsal head; and
manipulating and moving the first metatarsal head in the frontal, saggital, and transverse planes of the right foot, and effecting left counterclockwise rotation of the first metatarsal head with the surgical tool as the surgical tool is in tightened engagement with the first metatarsal head.

111. The method of performing bunion surgery of claim 110 wherein the positioning guide is a guide wire.

112. The method of performing bunion surgery of claim 110 wherein the positioning guide is a guide wire pre-embedded in the first metatarsal head at the bone boring position.

113. The method of performing bunion surgery of claim 110 wherein the positioning guide is 0.45 mm diameter k-wire.

114. The method of performing bunion surgery of claim 110 wherein an external drill is connected to the handling end of the tool body, the drill then being used to rotate the surgical tool around the main body axis.

115. The method of performing bunion surgery of claim 110 further comprising:
providing loops located at positions along the tool body; and
directing additional wires through said loops and using the additional wires to provide temporary fixation of bone material.

116. The method of performing bunion surgery of claim 110 further comprising:
providing an angulation tool guide having a positioning angle;
positioning the angulation tool with respect to the boring position; and
aligning the positioning guide with the angulation tool at the positioning angle and inserting the positioning guide into the first metatarsal head at the boring position.

117. A surgical method comprising:
providing a surgical tool having a tool body, the tool body having a main body axis, a threaded end, a handling end, and a guide hole extending through the tool body substantially along the main body axis, the threaded end having threads and a tool stop, the handling end being positioned to allow a user to grasp, manipulate, and rotate the tool body around the main body axis;
providing an angulation tool guide having a positioning angle and positioning the angulation tool guide near a bone boring position;
providing a positioning guide and aligning the positioning guide with the angulation tool at the positioning angle;
inserting the positioning guide into a bone at the bone boring position;
extending the positioning guide through the guide hole at least partially through the tool body;
aligning and guiding with the positioning guide the threads of the surgical tool to the bone boring position;
rotating the surgical tool in a boring direction around the main body axis to bore into and engage the bone with the threads at the bone boring position, the threads further tightening engagement with the bone as the tool body continues to rotate in the boring direction around the main body axis;
continuing to rotate the surgical tool until the tool stop contacts the bone to prevent the tool from being further rotated around the main body axis and to prevent further boring into the bone; and
manipulating, moving, and rotating the bone with the surgical tool while the tool is in tightened engagement with the bone.

118. The surgical method of claim 117 wherein the threads of the tool body are rotated around the main tool body axis in a right, clockwise direction to bore into and engage the bone.

119. The surgical method of claim 117 wherein the threads of the tool body are rotated around the main tool body axis in a left, counterclockwise direction to bore into and engage the bone.

120. The surgical method of claim 117 wherein the positioning guide is a guide wire.

121. The surgical method of claim 117 wherein the positioning guide is a guide wire pre-embedded in the bone at the bone boring position.

122. The surgical method of claim 117 wherein the positioning guide is 0.45 mm diameter k-wire.

123. The surgical method of claim 117 wherein an external drill is connected to the handling end of the tool body, the drill then being used to rotate the surgical tool around the main body axis.

124. The surgical method of claim 117 further comprising:
providing loops located at positions along the tool body; and
directing additional wires through the loops and using the additional wires to provide temporary fixation of bone material.
